(19) United States
(12) Patent Application Publication
(10) Pub. No.: US 2009/0234262 A1
(43) Pub. Date: Sep. 17, 2009

(54) Health Monitoring and Management System

(76) Inventors: Lawrence G. Reid, JR., Germanton, NC (US); Edward Grant, Raleigh, NC (US); Meghan Sarah Hegarty, Raleigh, NC (US); Frederick Livingston, Raleigh, NC (US)

Correspondence Address: KILPATRICK STOCKTON LLP, 1001 WEST FOURTH STREET, WINSTON-SALEM, NC, 27101, US

(21) Appl. No.: 12/402,623

(22) Filed: Mar. 12, 2009

Related U.S. Application Data
(60) Provisional application No. 61/036,122, filed on Mar. 13, 2008.

(51) Int. Cl. A61B 5/11; A61H 9/00

(52) U.S. Cl. 601/152; 600/595

(57) ABSTRACT

A health monitoring and management device, system, and/or method can include a sensor adapted to detect changes in one or more health indicators and transmit data related to the health indicators. The system can further include an interventional element adapted to receive a health intervention command and provide a health intervention related to the health indicators. The system can further include a microprocessor adapted to receive and analyze the health indicator data transmitted by the sensor, formulate the health intervention command related to the health indicator data according to pre-determined parameters, and transmit the health intervention command to the interventional element. The health intervention command can be transmitted to the interventional element within a clinically relevant time period.

### CROSS-REFERENCE TO RELATED APPLICATION

This application claims benefit of U.S. Provisional Patent App. No. 61/036,122, filed Mar. 13, 2008, which application is incorporated by reference herein in its entirety.

### FIELD OF THE INVENTION

The present invention relates to a health monitoring and management system. Such a health monitoring and management system may be useful for automatic monitoring of changes in a patient's condition and providing real-time interventions in response to those changes.

### BACKGROUND OF THE INVENTION

The velocity of blood flow is an important indicator of vascular efficiency. The velocity of arterial blood flow serves as an indicator of the efficiency of nutrient and oxygen delivery, and the velocity of venous blood flow serves as an indicator of the efficiency of waste removal. A decrease in the velocity of blood flow, particularly in venous blood flow, can increase the potential for formation of dangerous blood clots and lower leg swelling, which can lead to certain vascular pathologies. Compressive pressure applied on and/or near an area of lower leg swelling and/or decreased venous blood flow can improve blood flow and decrease the risk of resulting complications.

Such compressive pressure applied with a compression garment can be static compression or dynamic compression. In conventional compression garments, static compression can be provided by a single layer fabric or multiple layer fabrics that are designed to provide a single, constant level of compressive pressure on an anatomical structure, such as a leg. Such static compression garments can have disadvantages. For example, the amount of compressive pressure provided in static compression systems may vary over time due to yarn fatigue (which can cause stretched yarn) and swelling of the anatomical structure being compressed.

Some conventional dynamic compression devices can apply variable pressures at different locations on an anatomical structure. These dynamic compression devices often make use of pneumatically controlled compression bladders. Dynamic compression devices can also have disadvantages. For example, dynamic compression devices are often found to be uncomfortable due to quick changes in the amount of compressive pressure being delivered. In addition, pneumatic compression bladders require pumps, which can make the devices bulky, noisy, and require an external source of energy to operate. As a result, such dynamic compression devices may not be suitable for wearing by a patient. A further disadvantage of some conventional pump and sleeve compression devices is that they control compression levels based on patient status information that is old (or lags from real time) and/or without direct patent status data.

Thus, there is a need for a health monitoring and management system that can provide monitoring of health indicators and dynamic management of therapeutic interventions in response to the monitored health indicators in real time. There is a need for such a system that is easily wearable. There is a need for such a system that can operate wirelessly.

### SUMMARY

Some embodiments of the present invention can include a health monitoring and management device, system, and/or method. In some embodiments, the health monitoring and management system can include a sensor adapted to detect changes in one or more health indicators and transmit data related to the health indicators. The system can further include an interventional element adapted to receive a health intervention command and provide a health intervention related to the health indicators. In some embodiments, the system can further include a microprocessor adapted to receive and analyze the health indicator data transmitted by the sensor, formulate the health intervention command related to the health indicator data according to pre-determined parameters, and transmit the health intervention command to the interventional element.

Certain embodiments of the health monitoring and management system can further include a plurality of sensors, each sensor adapted to detect changes in a different one of the health indicators. Such an embodiment can further include a plurality of interventional elements, each interventional element adapted to provide a different health intervention related to one of the different health indicators. In some embodiments, the health intervention command can be transmitted to the interventional element within a clinically relevant time period. In certain embodiments, the pre-determined parameters comprise a control algorithm configured to automatically control formulation of the health intervention command and transmission of the command to the interventional element.

In some embodiments of the health monitoring and management system, the sensor can be attachable to, or integrated with, a garment. In certain embodiments, the microprocessor can be attached to, or integrated with, a garment. In some embodiments, the system can further include a computer, and the health indicator data detected by the sensor can be transmitted from the microprocessor to the computer. The computer can be adapted to receive and analyze the health indicator data transmitted by the microprocessor, formulate the health intervention command related to the health indicator data according to pre-determined parameters, and transmit the health intervention command to the interventional element. In particular embodiments, at least the sensor, the microprocessor, and the interventional element can communicate with each other wirelessly.

In certain embodiments, the system can further include a capability to learn patterns in an individual's health indicators monitored over time, predict health interventions based on those patterns, and formulate intervention commands based on those predictions in response to subsequent changes in the individual's health indicators. In certain embodiments, the system can further include a computer database in which the health indicator data for a plurality of persons is stored and analyzed, whereby intervention commands based on collective data in the database are determined for health indicators subsequently monitored in individuals.

In some embodiments, the sensor can comprise an electrical, mechanical, ultrasonic, acoustic, optical, or tactile sensor, or combination thereof. In some embodiments, the sensor can be adapted to detect changes in a person's body movements.

In an illustrative embodiment, the system can include an adjustable compressive pressure garment, and the health intervention comprises adjustment of the compressive pressure in the garment. Such an embodiment including an adjustable compressive pressure garment can include a first sensor comprising a blood flow sensing system and second sensor comprising an edema sensing system. The interventional element can comprise an air pump connected to a pneumatic compression stocking, and the health intervention can comprise adjustment by the air pump to the amount of air in the compression stocking related to the level of edema and blood flow detected.

Some embodiments can comprise a health monitoring system that includes a sensor adapted to detect changes in one or more health indicators and transmit data related to the health indicators, and a microprocessor adapted to receive, store, and transmit the health indicator data transmitted by the sensor.

In some embodiments, a health monitoring and management method can include detecting changes in one or more health indicators, and transmitting data related to the health indicators to a microprocessor, where the health indicator data can be analyzed. A health intervention command can be formulated related to the health indicator data according to pre-determined parameters, and the health intervention command can be transmitted to an interventional element. In certain embodiments of a method of health monitoring and management, the health intervention command can be transmitted to the interventional element within a clinically relevant time period related to the health indicators. In particular embodiments of such a method, the health intervention command can be automatically formulated according to pre-determined parameters and the intervention command transmitted to the interventional element by utilizing a control algorithm.

Features of a health monitoring and management device, system, and/or method may be accomplished singularly, or in combination, in one or more of the embodiments of the present invention. As will be realized by those of skill in the art, many different embodiments of a health monitoring and management device, system, and/or method are possible. Additional uses, advantages, and features of aspects of the present invention are set forth in the illustrative embodiments discussed in the detailed description herein and will become more apparent to those skilled in the art upon examination of the following.

### BRIEF DESCRIPTION OF THE DRAWINGS

FIG. 1 is a view of a health monitoring and management system in an embodiment of the present invention.

FIG. 2 is a diagrammatic illustration of a health monitoring and management system in another embodiment of the present invention.

FIG. 3 is blood flow velocity sensing system in an embodiment of the present invention.

FIG. 4 is an edema sensing system in an embodiment of the present invention.

### DETAILED DESCRIPTION

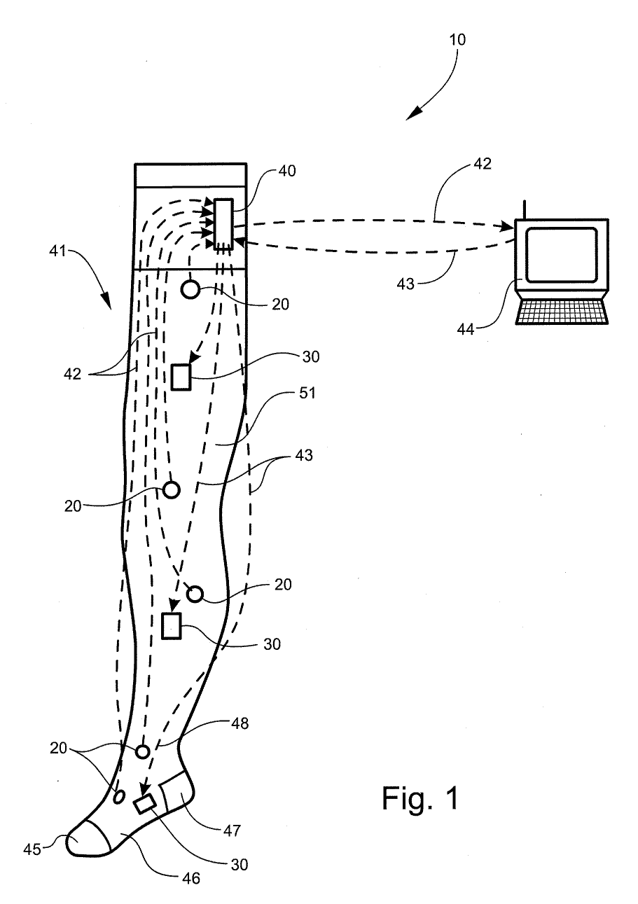

Some embodiments of the present invention can provide a health monitoring and management device, system, and/or method. FIGS. 1-4 illustrate various aspects of embodiments of such a health monitoring and management device, system, and/or method.

Some embodiments of the health monitoring and management system 10 can include one or more sensors 20 capable of monitoring external and/or internal conditions of a patient so as to detect changes in those conditions. In certain embodiments, a “sensor” 20 can be defined as having capability of monitoring and transmitting health indicator information. A sensor 20 can be adapted to detect some change in one or more health indicators. Health indicators include, for example, skin temperature, muscle activity, body motion, parameters related to musculoskeletal tissues, nerve conduction, blood flow, cardiac conductivity, cardiac output, respiratory activity, arterial and/or venous oxygenation levels, blood values such as blood chemistries, and the girth of a portion of an anatomical part (as an indicator of the volume of that anatomical part), among others.

Some embodiments can include both (a) sensor(s) 20 capable of monitoring and transmitting health indicator information and (b) interventional element(s) 30 capable of receiving health management intervention commands and providing intervention(s) related to those commands.

Some embodiments of the health monitoring and management system 10 can include a plurality of sensors 20. In this way, various combinations of health indicators can be monitored, and if desired, interventions related to the monitored indicators performed, in a single device and/or system. As a result of monitoring multiple health indicators simultaneously, the status of multiple body systems in a patient can be evaluated together. Accordingly, a more comprehensive view of a patient's overall clinical status can be ascertained, thereby allowing more accurately targeted interventions. The system 10 comprising a plurality of sensors 20 and interventional capabilities can thus have much greater functionality, efficiency, and efficacy than a conventional monitoring system in which a single health indicator is monitored.

The sensors 20 can be different types of sensors, including, for example, ultrasonic, acoustic, optical, and/or electrical sensors 20 to monitor different types of health indicator information. Any type of sensor 20 suitable for monitoring health indicators, or medical parameters, in a patient can be adapted for use in certain embodiments of the present invention. Such sensors 20 can utilize a sensing mechanism that monitors patient conditions in a non-invasive manner. For example, in certain embodiments, the sensor 20 can be configured to detect changes in a health indicator by contact with a patient's skin. In other embodiments, the sensor 20 can utilize one or more probes that can be placed in an internal body location for detecting changes in a health indicator.

The health monitoring and management system 10 can include various embodiments of a sensor 20, each sensor 20 configured to monitor a particular health indicator, for example, pulse rate or blood flow or another health indicator. In some embodiments, the sensor 20 can be adapted to monitor multiple health indicators. For example, an ultrasound sensor 20 that can typically be used to monitor blood flow may be adapted to also monitor bone density. Ultrasound waves impinging on bone produce a different and distinct wave shape, or signature, than the wave shape signature produced from ultrasound waves impinging on blood vessels. Thus, a single ultrasound sensor 20 can be adapted to monitor both blood flow and bone density.

Some embodiments of the health monitoring and management system 10 can include sensors 20 having the capability of monitoring a person's health status indicators, such as physiological measures, and/or behaviors over various periods of time. For example, the system 10 can be configured to monitor a person's exercise levels and patterns, as well as physiological responses to those behaviors, over a relatively short period of time, such as during a workout period. For instance, such a system 10 may be utilized to monitor whether an athlete is training at an optimal level, at a sub-optimal level, or is overtraining. Alternatively, or in addition, the system 10 can be configured to monitor a person's activities and physiological responses to those activities over a relatively long period of time, such as over several weeks of therapy. In this way, such a system 10 can monitor, record, analyze, and use information related to a person's activities and changes in health status indicators over time, including patterns of both health deterioration and health improvement.

Some embodiments of the health monitoring and management system 10 can include sensors 20 having the capability of monitoring a particular health indicator within pre-set ranges. For example, for tachycardic patients, the sensor 20 may be set to monitor only pulse rates above 100 beats per minute, and for bradycardic patients, the sensor 20 may be set to monitor only pulse rates below 60 beats per minute. Alternatively, a pulse rate sensor 20 may be set to monitor pulse rates either below 60 beats per minute, above 100 beats per minute, or both below 60 beats per minute and above 100 beats per minute. A sensor 20 for one health indicator can monitor that indicator at different intervals or within different ranges than a sensor 20 for a different health indicator. As an example, a sensor 20 configured to monitor pulse rate may be pre-set to measure pulse on a continuous basis, whereas a sensor 20 configured to monitor skin temperature may be pre-set to measure skin temperature only once every hour.

The combination of sensors 20 utilized for individual patients can be customized. For example, a particular patient may need sensors 20 to monitor both cardiac conductivity and oxygenation levels. The suite of sensors 20 incorporated into the health monitoring and management system 10 for that patient can include cardiac conductivity and oxygenation sensors 20, and may or may not include other sensors 20. For another patient who may need to have respiratory activity and body motion monitored, the suite of sensors 20 incorporated into the health monitoring and management system 10 for that patient can include those sensors 20 for respiratory activity and body motion, and may or may not include other sensors 20.

The design of a particular embodiment of the sensor 20 depends on the health indicator it is intended to monitor. Various embodiments of sensors 20 can utilize combinations of electrical, mechanical, acoustic, tactile, and/or other sensing mechanisms to monitor the intended health indicator. For example, one embodiment of the sensor 20 can include electrical components and may be configured to detect, for example, a change in flow of electrical current between two locations on a patient's body. Another embodiment of the sensor 20 can include mechanical components and may be configured to detect, for example, a change in movement of the patient. Another embodiment of the sensor 20 can include an ultrasound detection mechanism and may be configured to detect, for example, blood flow. Certain embodiments of an ultrasound sensor 20 can provide the advantage of monitoring vascular blood flow while a person is moving around, whereas conventional monitoring devices, such as an ultrasonic Doppler device require a person to remain still during monitoring. In some embodiments of the system 10, two or more of various types of sensing mechanisms can be utilized.

The sensor 20 preferably comprises a sensing capability that is sufficiently sensitive to detect desired changes in the particular health indicator it is monitoring. As examples, a blood flow sensor 20 can have a sensitivity appropriate to detect clinically important increments of change in blood flow, such as in volume of flow in a pre-set time; an oxygenation sensor 20 can have a sensitivity appropriate to detect change in percentage of blood oxygen saturation; and a temperature sensor 20 can have a sensitivity to detect a change in each tenth of a degree of temperature. Other sensors 20 can have a sensitivity appropriate to detect clinically important increments of change in the health indicator being monitored. In certain embodiments, the sensitivity of the sensor 20 can be adjusted, depending on desired thresholds or ranges for measurements for the particular health indicator(s). Desired thresholds or ranges for measurements of a particular health indicator can vary depending on the clinical status of a particular patient and the data needed to determine optimal interventions.

In some embodiments, the sensor 20 may include a display mechanism (not shown) to display changes in the health indicator data being monitored. As an example, the sensor 20 may include a light emitting diode (LED) display that changes in illumination intensity, frequency of blinking, color, or some other indication correlating to a change in health indicator being monitored. To illustrate, a pulse rate sensor 20 having an LED indicator can begin to blink when the patient's pulse exceeds a pre-set threshold, such as 100 beats per minute. As the patient's pulse rate continues to increase above 100 beats per minute, the LED indicator can blink at an increasingly faster rate. Likewise, when the patient's pulse decreases, the LED indicator can blink at a progressively slower rate. Such a visual display can provide the patient and/or another observer such as a caretaker with immediate qualitative feedback regarding the patient's pulse rate. In certain embodiments, the sensor 20 can include other types of visual indicators of changes in health indicator(s) being monitored. For example, in certain embodiments of the health monitoring and management system 10, the sensor(s) 20 can display the actual data being collected, such as pulse rate, skin temperature, or other health indicator.

In some embodiments, raw health indicator data collected by the sensor(s) 20 can be transmitted to a microcontroller, or microprocessor, 40 which can organize the data in one or more ways. For example, in one exemplary embodiment, one sensor 20 may collect data indicating the intensity of body motion at specific times. Another sensor 20 may collect data indicating pulse rate at specific times, and yet another sensor 20 can collect data indicating respiratory rate at specific times. From data collected by the sensor(s) 20, the microprocessor 40 may relate changes in pulse rate and/or respiratory rate to the intensity of body motion over a period of time. The related data can be sorted in an organized manner, for example, into a cardiovascular response index. In another embodiment, one sensor 20 may collect data indicating blood flow, for example, in a patient's leg. Another sensor 20 may collect data indicating a change in volume, or edema, in the leg. The microprocessor 40 may relate the raw blood flow data and leg volume data in an organized manner to provide a peripheral blood flow index.

The microprocessor 40 can be attached to, or integrated with, the health monitoring and management system 10, which may be attached to a patient, for example, by being in or on a garment being worn by the patient. The monitored health indicator data can be analyzed at the level of the on-patient microprocessor 40. Health indicator data received by the microprocessor 40 from the sensor(s) 20 can be analyzed and used to manage a response to the monitored health indicator data by providing one or more clinical interventions. For example, as shown in FIG. 1, in an embodiment of the health monitoring and management system 10 comprising an adjustable compressive pressure stocking 41, a signal 42 comprising health indicator data from the patient wearing the stocking 41 can be transmitted to the on-patient microprocessor 40, where the data can be analyzed and a desired response, or health intervention, to the health indicator data, such increasing compressive pressure by a certain amount to enhance blood flow, can be formulated for that patient. A signal 43 comprising a health intervention command coded to effectuate the interventional response can then be transmitted from the microprocessor 40 to one or more interventional elements 30 within the system 10. The microprocessor 40 can thereby cause the compressive pressure to adjust in portions or in all of the compression stocking 41 according to the response formulated for the patient's most recent data. In this manner, compressive pressure(s) in the compression adjustable device 41 can be controlled in such as way as to be most medically beneficial and comfortable to the patient.

In other embodiments, health indicator data monitored by the sensor(s) 20 can be transmitted from the microprocessor 40 by means of the health indicator signal 42 to an off-patient computer 44 for “offline” analysis. At the offline computer 44, the data can be analyzed and a desired interventional response to the health indicator data can be formulated for that patient. As in the example above, the interventional response can be increasing compressive pressure by a certain amount to enhance blood flow. The signal 43 comprising a health intervention command coded to effectuate the interventional response can be transmitted from the offline computer 44 to the microprocessor 40 and then to one or more interventional elements 30 within the system 10. Alternatively, the health intervention command signal 43 may be transmitted directly from the computer 44 to the interventional element(s) 30 within the system 10. In this way, the offline computer 44 can cause the compressive pressure to adjust in portions or in all of the compression stocking 41 according to the formulated response.

In some embodiments, the health monitoring and management system 10 can detect various physiological changes in a patient and analyze those changes relative to predetermined parameters. A management response signal, or health intervention command signal, 43 can be transmitted from the computer 44 to the microprocessor 40 and then to the interventional element(s) 30, or directly from the computer 44 to an interventional element 30, from which therapeutic interventions can be effectuated. When measurements taken by the health monitoring and management system 10 are outside the predetermined parameters, the system 10 can provide interventions based on those measurements. For example, in an embodiment in which the system 10 is associated with the compressive pressure garment 41, the system 10 can control adjustments of the levels of compressive pressure applied by the entire garment 41 or by particular portions of the garment 41 (such as in the toe 45, foot 46, heel 47, ankle 48, calf 50, and/or thigh 51), depending on detection and analysis of certain health indicators outside predetermined parameters. For example, when the compression adjustable garment 41 is a compression stocking and the health monitoring and management system 10 detects that blood flow in the calf 50 area has decreased below a predetermined level and/or that the compressive pressure being applied in the ankle 48 area is less than that applied in the calf 50, the system 10 can automatically increase the compressive pressure in the ankle 48 area in order to improve blood flow in the calf area 50.

Physical and/or physiological data of a patient using an embodiment of the health monitoring and management system 10 can be collected and analyzed in real time with desired changes in therapeutic interventions made by the interventional element(s) 30, or management component(s), of the system immediately or within a clinically relevant time period. A “clinically relevant time period” is defined as the time period for intervening related to a monitored health indicator that is outside predetermined parameters and beyond which period the patient is likely to experience deterioration in that indicator and/or other indicators if the intervention is not provided. The “clinically relevant time period” can vary depending on the health indicator and the extent to which the indicator is outside the predetermined parameters. For example, the “clinically relevant time period” for intervening for moderately decreased blood flow in a leg may be one hour, while the “clinically relevant time period” for intervening for a sustained heart rate of 200 may be less than one minute.

In some embodiments, the microprocessor 40 and/or the computer 44 can provide control of monitoring mechanisms to adjust to various movements of the patient and positions in which the sensor 20 may be placed by the patient. For example, if a wearer of a garment in the health monitoring and management system 10 changes position from sitting to standing, walking, and/or lying down, the microprocessor 40 and/or computer 44 can automatically adjust the sensitivity of the sensors 20 and/or which sensors 20 are monitored at a particular point in time. In this way, the system 10 can monitor health indicators on an uninterrupted basis and account for some patient-initiated variables, thereby providing health care providers more complete and accurate information about the person's physiological status and health patterns.

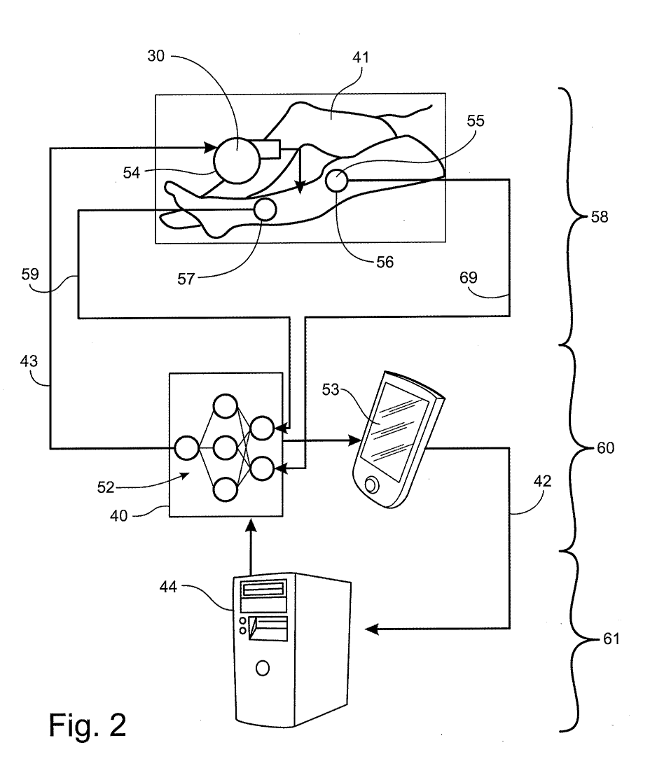

Some embodiments of the health monitoring and management system 10 can comprise a control system for automatically controlling interventions in response to health indicator measurements taken by the system 10. As shown in FIG. 2, such a control system may comprise an algorithm 52 programmed in the sensor 20, microprocessor 40, local electronic device 53, and/or computer 44. As an example, the control algorithm 52 for adjustment of compressive pressure in the compressive pressure stocking 41 can include commands for adjusting the compressive pressure provided by the stocking 41 depending on the volume, or girth, of the leg underneath the stocking 41 measured by the sensor 20. For example, if the girth of a leg changes as a result of a change in posture, the control system 52 can command activation of a pump 54 to increase/decrease pressure as needed to maintain the desired level of compression in the compressive pressure stocking 41.

Another example of a control system algorithm 52 is that for controlling patency of an arteriovenous fistula or dialysis shunt (together defined as “dialysis access route”). Such a control system algorithm 52 can include commands for activating a pump for flushing the dialysis access route when blood flow in the dialysis access route drops below a pre-set level. The control algorithm 52 can include commands for various levels of intervention. For example, if the sensor 20 detects that blood flow in the dialysis access route drops below a first level, the control system 52 can command a pump to flush the dialysis access route with a first solution, for example, a bolus of saline. If the sensor 20 detects that blood flow in the dialysis access route drops below a second, lower level, the control system 52 can command a pump to flush the dialysis access route with a second solution, for example, an anticoagulant flush. Alternatively, if after administration of the first solution the blood flow in the dialysis access route does not increase, the control system 52 can command a pump to flush the dialysis access route with a second solution, for example, an anticoagulant flush.

Another example of a control algorithm 52 is that for control of insulin delivery. Such a control system algorithm 52 can include commands for adjusting the rate of insulin being delivered by a pump depending on the blood sugar level measured by the sensor 20. For example, if a patient's blood sugar exceeds a pre-determined level as measured by the sensor 20, the control system 52 can command the insulin pump to deliver a certain amount of insulin to the patient. The control system algorithm 52 can comprise multiple levels of control related to the monitored health indicator. For example, for a first blood sugar level measured by the sensor 20, the control system 52 can command the insulin pump to deliver a first amount of insulin to the patient. If after an appropriate period of time following administration of the first dose of insulin, the blood sugar level monitored by the sensor 20 continues to exceed a pre-determined threshold related to expected blood sugar parameters following such a first dose, the control system 52 can command the insulin pump to deliver a second amount of insulin to the patient. Some embodiments of the health monitoring and management system 10 can comprise a control system 52 for controlling interventions in response to other health indicator measurements taken by the system 10.

In some embodiments, the sensor 20 can be self-contained. That is, the sensor 20 can comprise all components necessary to perform its intended function, such as sensing, collecting, and transmitting health indicator data. In some embodiments, the sensor 20 can be miniaturized. An exemplary embodiment of a sensor 20 can include an ultrasound sensing mechanism, a transmitter, and a battery. Such an embodiment may have dimensions of about ¼ inch by ¼ inch. In certain embodiments, the ultrasound sensor, transmitter, battery, and other electronic connections and/or components can be contained within a polymeric material poured about all of these components. In this way, the components can be protected against exposure to environmental variables. In addition, the sensor 20 can be disposable, so that when the battery life is exceeded, the sensor 20 can be replaced by another sensor 20. Such embodiments of self-contained and/or miniaturized sensors 20 can thus be easily worn by a patient. For example, the sensor 20 can be attached to, or integrated into, a garment. The garment may be one that is typically worn by a patient, such as an undergarment.

In certain embodiments of the health monitoring and management system 10, one or more sensors 20 can be attached to, or integrated into, a garment adapted to provide health management, or therapeutic, interventions. Thus, in certain embodiments, the health monitoring and management system 10 can further comprise a wearable therapeutic device. For example, sensors 20 can be attached to, or integrated into, the compressive pressure stocking 41, a wound dressing, a vest, an abdominal binder, a lymphedema sleeve, etc. For purposes of illustration, some embodiments of the health monitoring and management system 10 can include the compression adjustable garment 41 and have the capability of changing or adjusting the compressive pressure of selected portions of the garment 41 or of the entire garment 41 while the garment 41 is being worn. Changing the compressive pressure of the garment 41 can help manage vascular flow in an anatomical structure underneath the garment 41.

In certain embodiments, self-contained and/or miniaturized sensors 20 can be modular, such that the sensors 20 can be placed at various desired locations on a patient, such as at different locations in or about a garment. The sensor(s) 20 for monitoring a particular health indicator may be attached to specific locations on a garment so as to provide measurements from critical points on a patient's body. Multiple ones of a certain sensor 20 can be placed at various locations on a garment so as to provide a profile of measurements for the particular health indicator being monitored.

In certain embodiments of the present invention, the health monitoring and management system 10 can comprise electronic components integrated into the fabric of a wearable system. For example, a wearable health monitoring and management system 10 can include components such as electronic circuits, resistors, capacitors, and coils made from conductive yarns or other materials. A material's response to changes in pressure, humidity, temperature, or other conditions can be measured by observing a textile electronic element's response to a finite impulse of voltage or current. The response of the electronic element can be analyzed to determine changes in impedance, capacitance, and/or inductance of the element. In a particular illustrative embodiment, the health monitoring and management system 10 can include sensor(s) 20 that can gather arterial and venous blood flow information using continuous wave ultrasonic and body impedance feedback. Bio-impedance analysis techniques may be utilized to analyze and manage particular health conditions, such as lower leg swelling. In certain embodiments, such a bio-impedance system adapted to monitor and manage lower leg swelling can be a stand-alone system that is wearable. The “smart” or “intelligent” fabric of such a health monitoring and management system 10 can utilize combinations of such electrical components to provide sensors 20 that can sense a variety of behavior and health indicators and microcontrollers 40 that can allow use of interactive digital devices with the garment.

In certain embodiments, the sensor 20 may be an electrically passive device, or an integrated device, with measurement and transmission capability. For example, a garment comprising the biomedical sensor(s) 20 can include electrical power distribution and data transmission capabilities. Such a garment can further include a coupling circuit for allowing contactless transmission of power and data between sensors 20 and a circuit external to the garment.

In some embodiments, the sensor 20 can operate in a wireless manner, as illustrated in FIG. 1, for example. That is, the sensor 20 can wirelessly transmit collected health indicator data to the microprocessor 40. In embodiments in which the sensor 20 incorporates the interventional element 30, the sensor 20 can wirelessly receive health intervention commands. In certain embodiments, the sensor 20 can comprise the microprocessor 40 within the sensor 20. In this way, the sensor 20 can wirelessly transmit collected health indicator data directly to a database and/or to a computer 44, or other appropriately configured electronic device, at a location remote from the patient, such as at a hospital or clinic.

In other embodiments, the microprocessor 40 can be included in an appropriately configured electronic device separate from the sensor 20 that is attached to a desktop or laptop computer 44, or to a local electronic device 53 such as a personal digital assistant (PDA) or “smart phone” equipped with an appropriate software application. FIG. 2 shows an example of a local electronic device 53. In this embodiment, the microprocessor 40 and control algorithm 52 can be separate from the local electronic device 53. In other embodiments, the local electronic device 53 can include the microprocessor 40 and control algorithm 52 incorporated into the device 53. The electronic device 53 can be located at the patient's location, such as in the patient's home. The electronic device 53 can transmit the collected health indicator data to the computer 44, a designated database, and/or to a healthcare practitioner. In some embodiments, the microprocessor 40 and/or electronic device 53 can have a capability to provide local data storage. The electronic device 53 may be able to transmit health indicator data and/or receive health intervention commands either in a wired or wireless manner. In certain embodiments, the local electronic device 53 can be a remote transmission device that can be worn by the patient, for example, on a belt.

Embodiments of the health monitoring and management system 10 can be utilized with patients in a healthcare setting, such as a clinic, hospital, or long-term care facility. In this way, a healthcare practitioner can directly observe a patient while also receiving health indicator data collected by the sensor(s) 20. In addition, embodiments of the system 10 can be utilized with patients in settings remote from a healthcare practitioner. For example, the system 10 having one or more sensors 20 and/or interventional elements 30 can be worn by a patient while at home, at work, or in other locations, and the health indicator data detected by the sensor(s) 20 can be transmitted to a remote site, such as a hospital or clinic, where a healthcare practitioner can receive the transmitted data and provide therapeutic intervention commands to the system, if desired. Embodiments of such a system can provide real-time health indicator monitoring and management.

In certain embodiments, the health indicator data monitored for an individual patient can be utilized by a software program to “learn” patterns in that patient's health status over time. For example, if the venous blood flow in a patient's calf 50 area decreases by approximately the same amount each time the patient moves from a sitting to a standing position, the software program can “learn” that pattern of change and predict that the compression stocking 41 being worn by the patient should have a certain calculated amount of increase in compressive pressure below and/or in the calf 50 area for each subsequent time the patient stands. Such “data mining,” or “machine learning” can allow the health monitoring and management system 10 to provide quicker and more accurate and effective responses to changes in a particular patient.

In certain embodiments, the health monitoring and management system 10 can further include the collection of health indicator data from groups of patients into a database. The “offline” computer 44 can be programmed to analyze and learn patterns of health indicator data related to certain patient behaviors for clinically relevant samples of patients and/or entire populations of patients. To illustrate hypothetically, collections of data from a large sample of patients may reveal, for example, that in 80 percent of male patients over age 65 having Type II diabetes and who weigh over 220 lbs., for those who have a venous stasis pressure ulcer on the heel 47 or ankle 48, venous blood flow in the heel 47 or ankle 48 drops on average by 20 percent when the patients move from a sitting to standing position. The program may also have learned, as a hypothetical illustration—from storing real-time data related to patient management interventions by the system—that increasing the compressive pressure by 30 percent on the foot 46 of those same patients when they move from a sitting to standing position causes the venous blood flow to return to the sitting rate within one minute. This type of patient information data collection, storage, and analysis can allow the health monitoring and management system 10 to provide more effective and reliable care for groups of patients.

The “offline” computer 44 may be a stand-alone computer 44 or may be connected to a computer network. The network connection may be accomplished by physically connecting a cable from the monitoring device to a terminal connected to the network. Alternatively, the health monitoring system network connection can be wireless. The network can be a private networked system, such as a network operated by a hospital or clinic. In certain embodiments, the network database can be an internet web site. The internet site can be a proprietary site in which confidentiality of patient information can be maintained. Uploading monitored patient data onto a network database can allow long-term tracking of an individual patient's health patterns, as well as cumulative researchable data for particular patient populations.

In certain embodiments of the system 10 comprising only the sensor(s) 20, the system 10 can be utilized to gather health indicator data from a particular patient and store that data for later use. Such an embodiment of the system 10 can further include the microprocessor 40 adapted to receive, store, and transmit the health indicator data transmitted by the sensor. Because of these capabilities, such an embodiment of the monitoring system 10 in a garment can be known as a “smart sleeve.” For example, an embodiment of a sensor-only system 10 can be utilized to monitor a first set of health indicator data for a patient at a first time point, the data can be stored within the system 10 or externally in a data storage device such as a computer, and a second and subsequent sets of health indicator data can be monitored for the patient at a second and subsequent time points. The health indicator data for the patient gathered at the first time point can be a baseline of clinical information against which the second and subsequent sets of health indicator data can be compared. In this way, changes in the clinical status of the patient can be evaluated over various time periods.

As an example of how an embodiment of a sensor-only system 10 can be utilized, having health indicator data for a patient available at different time points can allow a clinician, such as a physician, evaluate changes in clinical status of the patient without any interventions over time or in response to one or more interventions. To illustrate, a patient's health indicator data can be monitored on four different dates. Selected health indicators monitored on the first monitoring date can provide a baseline of clinical data. After an appropriate interval related to the health indicators being monitored, the same health indicators can be monitored on a second monitoring date. During the interval between the first and second monitoring dates, there may be no intervention related to the monitored health indicators provided to the patient. Thus, a comparison of the health indicator data monitored on the first and second monitoring dates can provide an indication of the patient's change in clinical status without any purposeful therapeutic intervention. Following the second monitoring date, a first therapeutic intervention related to the health indicators being monitored can be provided to the patient. Then, on a third monitoring date at an interval following the first intervention sufficient to allow for a clinical response from the first intervention, the health indicators can be monitored again. Likewise, following the third monitoring date, a second therapeutic intervention related to the health indicators being monitored can be provided to the patient. Then, on a fourth monitoring date at an interval following the second intervention sufficient to allow for a clinical response from the second intervention, the health indicators can be monitored again. In this way, responses in the patient's health indicators can be evaluated with respect to no intervention and to both the first and second interventions. Accordingly, by monitoring responses to different interventions, the most effective interventional modality can be determined for a patient.

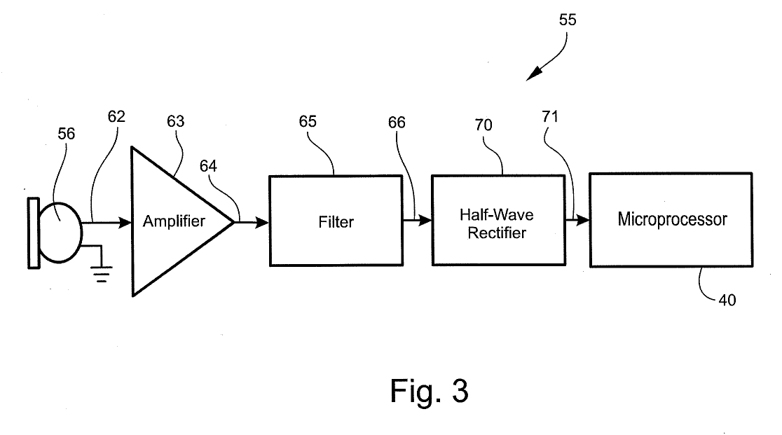

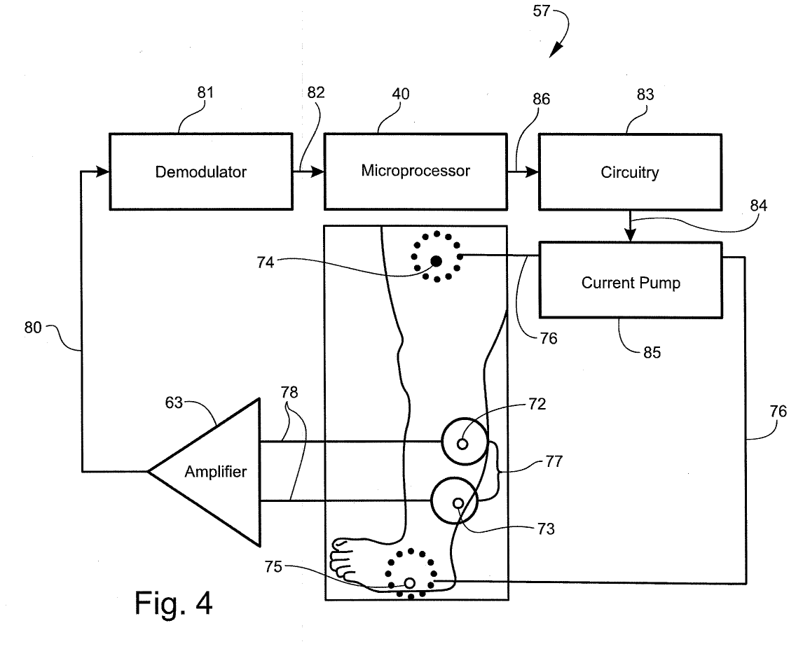

FIGS. 2-4 illustrate aspects of an exemplary embodiment of the health monitoring and management system 10. As shown in FIG. 2, the adjustable pneumatic compression stocking 41 can have the pump 54, such as a miniature diaphragm pump, connected to the compression stocking 41. A blood flow sensing system 55, an example of which is illustrated in FIG. 3, can be connected to the compression stocking 41. The blood flow sensing system 55 comprising a microphone sensor 56 can detect blood flow velocity in the leg of a person wearing the compression stocking 41. In addition, or alternatively, a lower leg volume, or edema, sensing system 57, an example of which is illustrated in FIG. 4, can be connected to the compression stocking 41. The edema sensing system 57 can sense and detect changes in edema, or swelling, in the leg of a person wearing the compression stocking 41. In some embodiments, as shown in FIG. 2, the compression stocking 41, blood flow sensing system 55, and edema sensing system 57 can collectively comprise a compression stocking network 58.

Health indicator data related to blood flow velocity and edema sensed by the blood flow sensing system 55 and edema sensing system 57, respectively, can be input to a data processor. The data processor can be, for example, the microprocessor (microcontroller) 40, or other integrated circuit possessing computing functionality. The input data can be processed through the control algorithm 52, for example, the compressive pressure control algorithm 52. In certain embodiments, when blood flow velocity and/or lower leg edema reach pre-set thresholds (or a single threshold for a combined profile of blood flow and edema values), the microcontroller 40 can control a system for changing the compressive pressure on the wearer's leg(s) provided by the compression stocking 41. For example, in an embodiment in which the compression stocking 41 includes the pump mechanism 54 for changing compressive pressure, the microcontroller 40 can send a control signal to actuate the pump 54 to increase or decrease the air pressure within the compression stocking garment 41 and thereby increase or decrease the compressive pressure on the wearer's leg(s).

In some embodiments, the health monitoring and management system 10 can further include the local electronic device 53, for example, a wireless communication device, as shown in FIG. 2. The wireless communication device 53 can be in communication with the microcontroller 40. In this manner, the wireless communication device 53 can capture and store on a local level the blood flow velocity and edema data sensed by the blood flow sensing system 55 and edema sensing system 57, respectively, and/or processed by the microprocessor 40. In addition, the wireless communication device 53 can capture and store data related to control of compressive pressures in the compression stocking 41 actuated by the microprocessor 40 in response to the sensed blood flow and edema data values. In certain embodiments, the microprocessor 40 can communicate with the wireless communication device 53 in a wireless manner. In other embodiments, the microprocessor 40 can be physically connected to the wireless communication device 53, such as with a cable. In some embodiments, as shown in FIG. 2, the microprocessor 40 and control algorithm 52 and the wireless communication device 53 can collectively comprise a local control and data storage system 60.

In some embodiments, the wireless communication device 53 can be in communication with the centralized computer 44 and database, as shown in FIG. 2. The central computer 44 and database can be in a location remote from the patient. In the embodiment in FIG. 2, the central computer 44 and database can be in a location remote from the compression stocking network 58 and the local control and data storage system 60. Thus, the computer 44 and database can comprise a remote data storage system 61. The remote computer and database system 61 can be utilized to store data transmitted from the local control and data storage system 60, for example, the monitored data and the management, or control and intervention, data captured by the wireless communication device 53. In certain embodiments, the central computer 44 and database can be further utilized for various purposes related to collected data. For example, the central computer 44 and database can be utilized to store data from a plurality of persons wearing one of the compression stockings 41. Such collective data can be processed to improve the control algorithm 52 for the health monitoring and management system 10, for example, to enhance intervention responsiveness and treatment results for individuals wearing the compression stocking 41. In addition, such collective data may be used for health research purposes, for example, related to lower leg edema and blood flow with respect to particular patient conditions and physical metrics.

FIG. 3 illustrates one example of the sensor 20, in particular, the blood flow sensing system 55 useful in some embodiments of the health monitoring and management system 10. Such a blood flow sensing system 55 can be adapted to monitor blood flow and can include the microphone 56 attached to the compression stocking 41, as shown in FIG. 2. Blood flow in the leg of a wearer of the compression stocking 41 can produce sound variations, depending on the velocity and quality of blood flow. The microphone 56 can sense such sound variations and create an acoustic signal 62 that is representative of those sounds. Acoustic signals 62 from the microphone 56 can be transmitted to an amplifier 63. An amplifier 63 is a device for converting a low energy signal into a higher energy signal, that is, for increasing the power or amplitude of an input signal. Accordingly, the amplifier 63 can convert low energy acoustic signals 62 input from the microphone into first, higher energy, or amplified signals 64. In some embodiments of the system, a fixed amplification can be applied if the characteristics of the incoming signal 62 are well-known, or a variable amplification can be applied to account for individual differences.

The first amplified acoustic signals 64 can then be transmitted to filtering circuitry 65. High pass filters can be used to pass frequencies above a specified “cutoff frequency” and attenuate, or reduce the amplitude of, signals with lower frequencies. Such high pass filters are useful for eliminating signal offsets (that is, dc-shift) which may result from constant background noise. Low pass filters can be used to pass frequencies below a specified “cutoff frequency” and attenuate signals with higher frequencies. Such low pass filters are useful for eliminating 50/60 Hz noise for other electronic sources. Bandpass filters can be created by combing high and low pass filters, and can be used in an embodiment of the acoustic blood flow monitoring system 55 to smooth the incoming acoustic signal 62 and extract useful signal components.

The filtered acoustic signals 66 representing blood flow velocity can then be transmitted to a half-wave rectifier 70. A half-wave rectifier 70 is an electrical circuit that can be used to block either the positive or negative portion of an alternating current (AC) signal. In this embodiment, the negative portion of the signal 66 is eliminated so that the signal 71 can be sampled by the analog-to-digital (ADC) module on a microprocessor 40. The microprocessor 40 can then process the acoustic data 71 through various computing functions. For example, the signals 71 received by the microprocessor 40 may be compared with normative blood flow velocities, compared with previous blood flow data for a particular person, stored in one or more databases including wirelessly transmitting data to a remote site, utilized to actuate a change in compressive pressure in the compression stocking 41, and/or further analyzed.

FIG. 4 illustrates an edema sensing system 57 useful in an embodiment of the health monitoring and management system 10. Such an edema sensing system 57 can include four electrodes at spaced-apart locations in the compression stocking 41. In such a system 57, an electrical current 76 can be transmitted across the outer pair of electrodes 74, 75 and recorded by the inner pair of electrodes 72, 73. For example, the two recording electrodes 72, 73 can be positioned in the calf 50 area, the current-originating electrode 74 placed in the thigh 51 area, and the current-terminating electrode 75 placed in the foot 46 area of the person. The change to the signal 76 as it passes between the current-originating and current-terminating electrodes 74, 75, respectively, is sensed by the recording electrodes 72, 73. This change 78 represents a change in impedance, or resistance to the flow of the electrical current 76. An increase in impedance can represent a decrease in edema in the person's leg (that is, the electrical resistance of a conductor varies inversely with volume).

With respect to the edema sensing system 57, changes 78 in impedance can be measured through application of Ohm's Law (i.e., Voltage=Current*Resistance): if a constant current 76 is applied to the leg, then a change 78 in impedance/resistance can be seen as a change 78 in voltage. As is shown in FIG. 4, a Howland current pump 85 can be used to provide a constant current source 76 regardless of the load attached. Other current sources may be utilized to provide the electrical current 76 for monitoring edema in certain embodiments of the present invention. Furthermore, in certain embodiments of the system, the frequency of the current source 85 can be varied using signaling commands 86 sent from the microprocessor 40 to specialized circuitry 83, for example, adjustable frequency sine wave circuitry.

In order to maintain safe levels of current 76 being applied to the body, the stimulating current 76 and recorded signals 78 can often be very small in amplitude. The recorded signal 78 can thus be amplified by an amplifier 63, and the amplified signal 80 passed through a demodulator 81. A demodulator 81 is an electronic circuit used to recover the information content from the carrier wave of the signal 78. In this embodiment, the demodulator 81 can recover, for example, the amplitude of the recorded signal 78 (i.e., which varies inversely with leg volume). The demodulated signal 82 can then be transmitted to a microprocessor 40 in which the signal 82 data can be processed through various computing functions. For example, the signals 82 received by the microprocessor 40 may be compared to signals representative of normative leg volume, compared with previous leg volume data for a particular person, stored in one or more databases including wirelessly transmitting data to a remote site, utilized to actuate a change in compressive pressure in the compression stocking 41, and/or further analyzed.

In another embodiment (not shown), the health monitoring and management system 10 may be adapted to detect nerve signals. The health monitoring and management system 10 may further include the capability of analyzing and creating a response to analysis of the detected nerve signals. In certain embodiments, the system 10 may translate detected nerve signals into operational signals. For example, the system may translate nerve signals into signal commands for operating a prosthetic limb.

Some embodiments of the health monitoring and management system 10 according to the present invention can provide advantages over conventional health monitoring and health intervention systems. For example, some embodiments of the present invention can provide both monitoring of a patient's health indicators and management of health indicator status by therapeutic interventions in a single device and/or system 10. The responses to changes in health indicators can occur in real time. As a result, such a system 10 can provide quicker and more accurate management of certain health conditions. Another advantage is that some embodiments of the present invention can provide a plurality of sensors 20 for monitoring various combinations of health indicators in a single device and/or system 10, thereby allowing a more comprehensive view of a patient's overall clinical status and more accurately targeting interventions. Another advantage is that some embodiments of the present invention can provide interventional elements 30 capable of intervening to managing the monitored health indicators within predetermined parameters.

Another advantage is that some embodiments of the present invention can provide health monitoring and management components in a device and/or system 10 that can be utilized remotely from a healthcare setting such as a hospital or clinic. Another advantage is that some embodiments of the present invention can provide a health monitoring and management system 10 that is wearable for extended periods and that is mobile and comfortable. Another advantage is that some embodiments of the present invention can provide a health monitoring and management system 10 that is capable of transmitting health indicator data and receiving health intervention data wirelessly. Another advantage is that some embodiments of the present invention can provide a health monitoring and management system 10 that is a stand-alone system.

The present invention can include embodiments of a method of making a health monitoring and management system 10. The present invention can include embodiments of a method of using a health monitoring and management system 10. Such methods of making and/or using the health monitoring and management system 10 can include aspects and features of various embodiments of the health monitoring and management system 10 as described herein.

For example, in some embodiments, a health monitoring and management method can include detecting changes in one or more health indicators, and transmitting data related to the health indicators to the microprocessor 40, where the health indicator data can be analyzed. A health intervention command can be formulated related to the health indicator data according to pre-determined parameters, and the health intervention command can be transmitted to the interventional element 30. In certain embodiments of a method of health monitoring and management, the health intervention command can be transmitted to the interventional element 30 within a clinically relevant time period related to the health indicators. In particular embodiments of such a method, the health intervention command can be automatically formulated according to pre-determined parameters and the intervention command transmitted to the interventional element 30 by utilizing a control algorithm 52.

Embodiments of the health monitoring and management device, system, and/or method can be utilized in a variety of applications. For example, some embodiments of the device, system, and/or method can be utilized with humans, while others may be utilized for monitoring and therapeutic purposes in animals. As described herein, some embodiments of the system 10 can be utilized to monitor health indicator data and/or manage therapeutic interventions related to the monitored data. Some embodiments of the system 10 can be utilized in care of wounds, either alone or in conjunction with other therapies. For example, the system 10 can include sensor(s) 20 adapted to detect changes in blood flow in a wound and can provide an intervention, such as a change in compressive pressure about the wound, in response to the blood flow health indicator data monitored by the sensor(s) 20. Some embodiments of the device, system, and/or method can be utilized to record changes in a patient's condition over time so as to document those changes for insurance purposes. In another application, health indicator data from a population of patients using an embodiment of the health monitoring and management device, system, and/or method can be stored in a common database. The collective data can then be used for research purposes, for example, to design parameters for therapeutic interventions across populations of patients.

Some embodiments of self-contained, miniaturized sensors 20 can be attached to, or integrated with, systems for monitoring and managing indicators other than those related to health. For example, a plurality of such sensors 20 may be molded in, or attached to, a motorized vehicle, such as an automobile, boat, train, submarine, or aircraft. Such sensors 20 may be configured to monitor various indicators related to the integrity and/or operation of such a vehicle. In one illustrative embodiment, such sensors 20 can be attached to, or integrated within, the skin of an aircraft to monitor the structural integrity, vibration patterns, or other engineering and/or performance indicators of the skin.

Features of a health monitoring and management device and/or system 10 and methods of making and/or using a health monitoring and management system 10 of the present invention may be accomplished singularly, or in combination, in one or more of the embodiments of the present invention. Although particular embodiments have been described, it should be recognized that these embodiments are merely illustrative of the principles of the present invention. For example, although the health monitoring and management system 10 of the present invention has been described herein in terms of embodiments including a compression adjustable stocking 41, such descriptions are for illustrative purposes only. It is contemplated that embodiments of the health monitoring and management system 10 of the present invention can comprise capabilities for monitoring various types of physical and health data other than blood flow and capabilities for managing various types of therapeutic interventions other than controlling compressive pressure in the stocking 41. Those of ordinary skill in the art will appreciate that a health monitoring and management system 10 and methods of making and/or using a health monitoring and management system 10 of the present invention may be constructed and implemented in other ways and embodiments. Accordingly, the description herein should not be read as limiting the present invention, as other embodiments also fall within the scope of the present invention.

What is claimed is:

1. A health monitoring and management system, comprising:
a sensor adapted to detect changes in one or more health indicators and transmit data related to the health indicators; and
an interventional element adapted to receive a health intervention command and provide a health intervention related to the health indicators.

2. The system of claim 1, further comprising a microprocessor adapted to receive and analyze the health indicator data transmitted by the sensor, formulate the health intervention command related to the health indicator data according to predetermined parameters, and transmit the health intervention command to the interventional element.

3. The system of claim 1, further comprising:
a plurality of sensors, each sensor adapted to detect changes in a different one of the health indicators; and
a plurality of interventional elements, each interventional element adapted to provide a different health intervention related to one of the different health indicators.

4. The system of claim 1, wherein the health intervention command is transmitted to the interventional element within a clinically relevant time period.

5. The system of claim 2, wherein the predetermined parameters comprise a control algorithm configured to automatically control formulation of the health intervention command and transmission of the command to the interventional element.

6. The system of claim 1, wherein the sensor is attachable to, or integratable with, a garment.

7. The system of claim 2, wherein the microprocessor is attachable to, or integratable with, a garment.

8. The system of claim 2, further comprising a computer, wherein the health indicator data detected by the sensor is transmittable from the microprocessor to the computer.

9. The system of claim 8, wherein the computer is adapted to receive and analyze the health indicator data transmitted by the microprocessor, formulate the health intervention command related to the health indicator data according to pre-determined parameters, and transmit the health intervention command to the interventional element.

10. The system of claim 2, wherein at least the sensor, the microprocessor, and the interventional element communicate with each other wirelessly.

11. The system of claim 1, further comprising a capability to learn patterns in an individual's health indicators monitored over time, predict health interventions based on those patterns, and formulate intervention commands based on those predictions in response to subsequent changes in the individual's health indicators.

12. The system of claim 1, further comprising a computer database in which the health indicator data for a plurality of persons is stored and analyzed, whereby intervention commands based on collective data in the database are determined for health indicators subsequently monitored in individuals.

13. The system of claim 1, wherein the sensor comprises an electrical, mechanical, ultrasonic, acoustic, optical, or tactile sensor, or combination thereof.

14. The system of claim 1, wherein the sensor is adapted to detect changes in a person's body movements.

15. The system of claim 6, wherein the garment comprises an adjustable compressive pressure garment, and
wherein the health intervention comprises adjustment of the compressive pressure in the garment.

16. The system of claim 1, further comprising a first sensor comprising a blood flow sensing system and second sensor comprising an edema sensing system,
wherein the interventional element comprises an air pump connected to a pneumatic compression stocking, and
wherein the health intervention comprises adjustment by the air pump to the amount of air in the compression stocking related to the level of edema and blood flow detected.

17. A method, comprising:
detecting changes in one or more health indicators;
transmitting data related to the health indicators to a microprocessor;
analyzing the health indicator data in the microprocessor;
formulating a health intervention command related to the health indicator data according to predetermined parameters; and
transmitting the health intervention command to an interventional element.

18. The method of claim 17, wherein transmitting the health intervention command to the interventional element further comprises transmitting the health intervention command within a clinically relevant time period related to the health indicators.

19. The method of claim 17, wherein formulating a health intervention command according to predetermined parameters further comprises automatically controlling formulation of the health intervention command and transmission of the command to the interventional element with a control algorithm.

20. A health monitoring system, comprising:
a sensor adapted to detect changes in one or more health indicators and transmit data related to the health indicators; and
a microprocessor adapted to receive, store, and transmit the health indicator data transmitted by the sensor.
